(19) United States
(12) Patent Application Publication
(10) Pub. No.: US 2016/0354074 A1
(43) Pub. Date: Dec. 8, 2016

(54) Orthopedic Screw with Suture Anchor and Multipurpose Connector

(71) Applicant: Stuart D. Miller, Owings Mills, MD (US)

(72) Inventor: Stuart D. Miller, Owings Mills, MD (US)

(21) Appl. No.: 14/728,784

(22) Filed: Jun. 2, 2015

(51) Int. Cl. A61B 17/04; A61B 17/86

(52) U.S. Cl. CPC A61B 17/0401; A61B 17/86; A61B 17/864; A61B 17/8605; A61B 17/8615; A61B 2017/044; A61B 2017/0462; A61B 2017/8655; A61B 2017/0459

(57) ABSTRACT

Bone screws with threaded screw heads that are designed to sit at or below the level of bone include features that allow them to be used to anchor sutures, either directly at the head of the screw or immediately adjacent to it. In particular, the screw head may have a hole or a pair of holes in which a bar or clip can be seated. The bar or clip carries structure that is adapted to retain a suture. Holes in the screw head can also be used to directly retain a bunching-type anchored suture, and can be threaded to retain other types of fasteners. Angled holes may be provided to allow an anchored suture to emerge adjacent to the screw head. Also disclosed are tools and methods for making holes in bone and tissue that are contiguous with the anchoring holes in the head of a surgical screw.

### BACKGROUND OF THE INVENTION

### 1. Field of the Invention

In general, the invention relates to medical devices and fasteners, and more particularly, to an orthopedic screw with a suture anchor and multipurpose connector.

### 2. Description of Related Art

In orthopedic surgery, screws are among the primary fasteners that are used to fix pieces of broken bone together and to secure other types of corrective hardware to bone. As with screws used in more prosaic applications, orthopedic screws generally have a shaft, at least a portion of which is threaded, and a head or cap, into which a driving tool can be inserted to drive the screw. Orthopedic screws vary widely in their size and other characteristics, depending on whether they are designed to insert into cortical or cancellous bone, the locations in which they are intended to be used, and the types of hardware they are intended to secure, if any. Some orthopedic screws have a threaded head which is designed to be inserted to or below the level of the bone cortex.

It is often necessary to attach soft tissue to bone. In these circumstances, suture anchors typically are used. One type of suture anchor has at least some characteristics of a screw, in that it has threads that cut into bone, but provides structure to which a suture can be attached. Once the suture anchor is inserted into bone, a suture can be attached to it. Some suture anchors come with a suture pre-attached.

Another type of suture anchor comprises suture material in a collapsible sleeve that balls up on activation and cannot be pulled through a hole, thus providing a fixation point. Still other suture anchors have spring arms which open after insertion into bone, thus preventing pull-out and providing a firm fixation point for a suture. Some anchors and/or anchor constructs are now used to hold bone to bone, with a fixation point in each side and a flexible suture in between, holding the bones in approximation until soft tissues heal and support the joint structure.

U.S. Patent Application No. 2012/0041484 to Briganti et al. discloses a typical form of screw-based suture anchor: a short, broad screw with a partial cannula or channel along its central opening. A clip inserts into the channel. The head of this anchor sits above the level of the bone, and the screw itself is adapted only to provide securement for the suture; it does not have sufficient length or other characteristics necessary to secure two bony structures together.

There are a number of other issues with typical orthopedic screws. For example, many orthopedic screws, especially cannulated screws, are easily stripped, making it difficult to extract screws later, or to replace them if a procedure requires revision.

### SUMMARY OF THE INVENTION

Aspects of the invention relate to surgical screws and to structures and methods for anchoring sutures and other elements to and within those screws. One particular aspect of the invention relates to a surgical screw and suture anchor. The screw has sufficient length and other characteristics to fix two structures together, and is typically of the so-called “headless” type, in which the head is threaded and intended to sit at or below the level of the bone. This type of screw head may be provided with additional features that allow it to serve as a suture anchor. In particular, the head of the screw includes a pair of holes opposite one another in the sidewall. These holes may be through-holes that open to both the interior and exterior faces of the sidewall, or they may be partial holes, open only to the interior face of the sidewall. The holes allow the screw to be used as a suture anchor.

In one embodiment according to this aspect of the invention, a bar with spring-biased pin ends inserts into the head of the screw, and the ends seat in the holes. The bar carries structure, such as ring structure, that is adapted to anchor a suture.

In another embodiment according to this aspect of the invention, a clip with a horizontal bar and two depending, pronged ends seats in the holes. The bar carries structure, such as ring structure, that is adapted to anchor a suture.

Another aspect of the invention relates to a surgical screw and suture anchor adapted to anchor a bunching-type anchoring suture. The screw has sufficient length and other characteristics to fix two structures together, and is of the “headless” type, in which the head is fully threaded and is adapted to sit at or below the level of the bone. The head of the screw has at least one angled hole in its sidewall that opens along both the inner and outer faces of the sidewall. The hole typically has a positive slope. In embodiments according to this aspect of the invention, a bunching-type anchoring suture is inserted through the hole to the inner face of the head, such that it rests in the socket of the screw head. When tension is applied to the suture, it bunches to a diameter greater than the diameter of the hole, thus causing it to be anchored within the screw. A hole of this type can also be used to secure other types of anchored sutures.

In embodiments according to this aspect of the invention, holes or openings in the head of the screw may be used to anchor or secure other structures, including screws, pins, and other fastening elements. To this end, the hole or holes in the head of the screw may be threaded.

Yet another aspect of the invention relates to tooling and methods for creating holes in surrounding bone and tissue that are contiguous with holes provided in the head of a screw. In one embodiment, a tool for creating such holes includes a plug, an arm or bracket, and a guide member. The plug inserts into and seats within the socket of a screw head and is typically shaped like a driving tool for the screw. The arm or bracket is connected to the upper end of the plug, and the guide member is supported by the bracket so that it is positioned in the same place and at the same angle as one of the holes, so that a drill bit or other hole-making tool passed through the guide member will create a hole that is contiguous with and extends at the same angle as one of the holes in the screw head.

Other aspects, features, and advantages of the invention will be set forth in the following description.

### BRIEF DESCRIPTION OF THE DRAWING FIGURES

The invention will be described with respect to the following drawing figures, in which like numerals represent like figures throughout the drawings, and in which:

FIG. 1 is a perspective view of an orthopedic screw and suture anchor according to an embodiment of the invention;

FIG. 2 is a top plan view showing the head of the screw of FIG. 1;

FIG. 3 is a cross-sectional view of the screw of FIG. 1, shown inserted into bone with a suture attached;

FIG. 4 is an enlarged cross sectional view of a portion of FIG. 3, illustrating the manner in which suture anchoring structure is secured within the head of the screw;

FIG. 5 is a cross-sectional view similar to the view of FIG. 4, illustrating an alternate mechanism for securing suture anchoring structure within the head of the screw;

FIG. 6 is cross-sectional view of the head of a screw according to another embodiment of the invention that provides anchor holes for use with a bunching-type soft anchor;

FIG. 7 is a cross-sectional view of the head of the screw of FIG. 6, illustrating its use with a different type of suture and anchor;

FIG. 8 is a partially cross-sectional view illustrating the use of a guide member that secures into the head and allows the user to drill a hole contiguous with the anchor holes of the screw;

FIG. 9 is a cross-sectional view of the head of a screw according to another embodiment of the invention, shown installed in bone, and illustrating structure allowing a second member or screw inserted through the head of the screw; and

FIG. 10 is a cross-sectional view of the head of a screw according to yet another embodiment of the invention, shown installed in bone, and illustrating arms or pins, mounted in a set of holes in the screw head, that are spring-biased to positively engage a screw driving tool.

### DETAILED DESCRIPTION

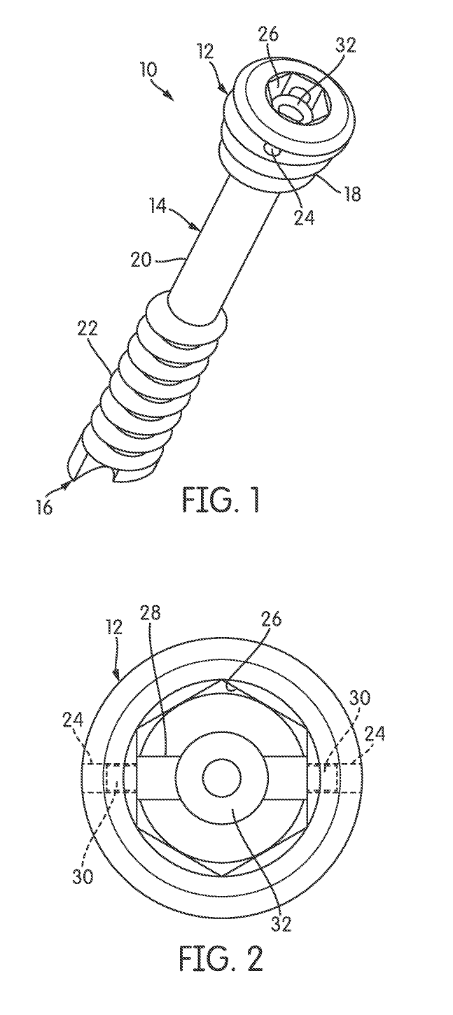

FIG. 1 is a perspective view of a screw, generally indicated at 10, according to one embodiment of the invention. The screw 10 is particularly adapted for use as an orthopedic bone screw, and its use for that purpose will be described in greater detail below. The screw 10 has a head 12, a shaft 14 attached to the head, and a tip 16 at the far end of the shaft 14.

Depending on the embodiment and the structures that it is intended to secure, the screw 10 may be of any length necessary to secure two structures together. It may be threaded along its entire length, or along only a portion of the length of the shaft 14. The precise characteristics of the tip 16 and any threads may vary, depending on the application and the nature of the structures that the screw 10 is to secure. In the illustrated embodiment, the head 12 is broader than the shaft 14 and is fully threaded with a first set of threads 18, making it a so-called “headless” screw that is adapted to sit at or below the surface of the bone.

The head 12 tapers down into the shaft 14. As was noted above, the shaft 14 of the screw 10 may be threaded along is entire length, or along only portions of its length. In the illustrated embodiment, a portion 20 of the shaft 14 is unthreaded, with a set of threads 22 proximate to the tip 16. The screw 10 may be made of any material of which screws 10 can be made, depending on the embodiment and the application. Typically, the material will be biocompatible and could, for example, be titanium, aluminum, surgical stainless steel, or a biocompatible polymer. The screw 10 of the illustrated embodiment has the general shape and characteristics of a CHARLOTTE® multi-use compression screw (Wright Medical Technology, Inc., Memphis, Tenn., USA), although screws 10 according to embodiments of the invention may have many different shapes and sizes.

When provided with appropriate features to allow attachment, the screw 10, and other “headless” (threaded-head) types of screws can serve as suture anchors, allowing a suture to be attached at the head 12 or, as will be described below in more detail, adjacent to the head 12. In the illustrated embodiment, the head 12 of the screw 10 includes a pair of holes 24, one on each side of the head 12, that extend generally horizontally from the outside to the inside of the head 12, terminating in the socket 26 of the head. In some cases, these holes 24 may be blind, opening into the socket 26 but not extending to the exterior of the head 12. However, in most embodiments, it will be useful to have holes 24 that open at both ends. The holes 24 allow suture anchor structure to be inserted and secured in the head 12 once the screw 10 is in place. Thus, the holes 24 allow the screw 10 to be used as a suture anchor. One advantage of the present invention is that many different types of screws are useable in embodiments of the invention if they are provided with simple features like the holes 24, as will be explained in greater detail below.

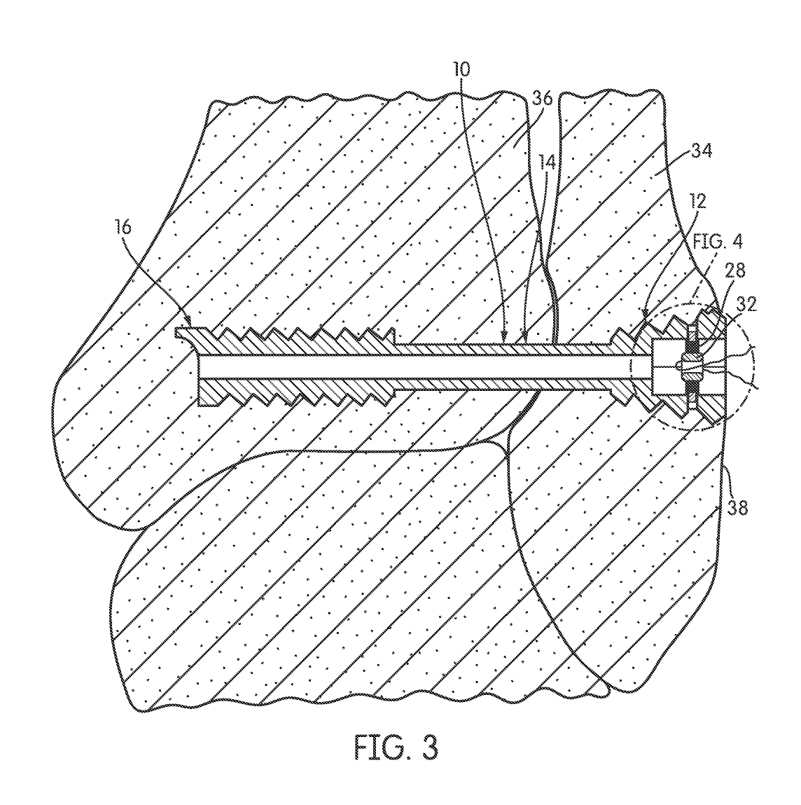

FIG. 2 is a top plan view of the head 12 illustrating the suture anchor structure in more detail. A bar 28 spans the socket 26 and has spring-loaded end pins 30 that insert into the holes 24. At its center, the bar 28 forms a ring 32, around which a suture may be tied.

The method by which the bar 28 and ring 32 are used to secure a suture are shown in more detail in FIG. 3, a cross-sectional view illustrating the screw 10 in place securing two pieces of bone 34, 36 together. The screw 10 spans both pieces of bone 34, 36, with the head 12 in one piece of bone 34 and the distal portion and tip 16 secured within the other piece of bone 36. As shown in FIG. 3, the screw 10 is set such that the top of the head 12 is generally level with the surface 38 of the bone 34. Thus, the screw 10 differs from a traditional suture anchor in that it secures two pieces of bone 34, 36 together in addition to its suture anchoring function. Typically, the bar 28 would be inserted after the screw 10 is driven into the position illustrated in FIG. 3.

In the illustrated embodiment, the screw 10 is fully cannulated, having a continuous passageway 13 through the screw 10 from the head 12 to the tip 16. Cannulation allows for access to the tissues beyond the screw 10 by inserting tools, needles, wires, etc., through the passageway 13 in the screw 10. Not all embodiments of the invention need be cannulated.

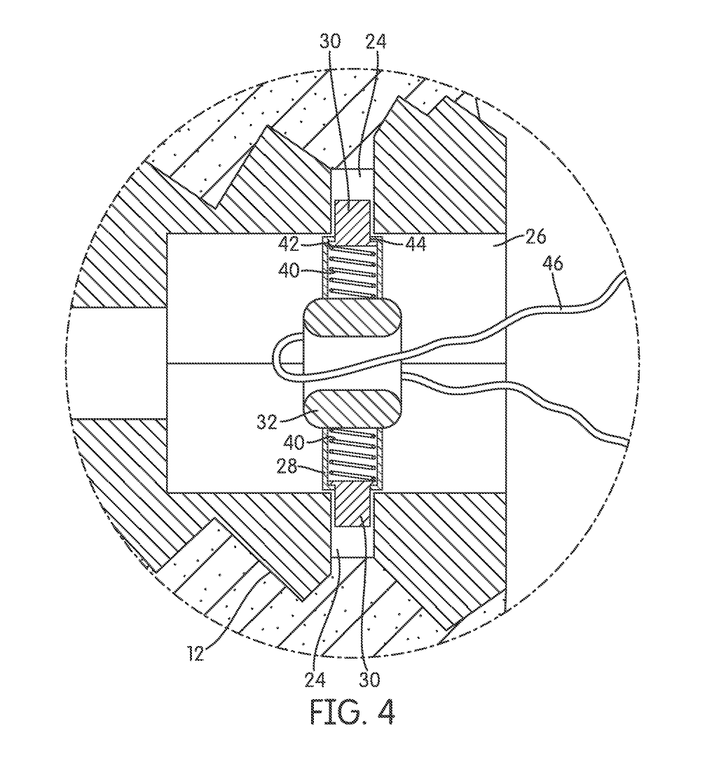

FIG. 4 is an enlarged cross-sectional view, illustrating the indicated portion of FIG. 3 in greater detail. The bar 28 is hollow, and has a main portion with a width almost equal to the width of the socket 26. A pair of small springs 40 are positioned within the bar 28 and bias the end pins 30 outwardly. Each end pin 30 has a small flange 42 that bears against the inner face of an inwardly-extending lip 44 of the bar 28 to retain one within the other. A suture 46 is looped around the ring 32.

One advantage of the invention is that essentially any conventional screw can become a screw useable in embodiments of the invention if it is provided with appropriate holes 24 or openings similar to those shown here. A variety of different structures may be inserted into those holes 24 or other openings to anchor sutures. For example, in some embodiments, a bar may be inserted into the holes that, instead of having end pins 30, is simply flexible and can flex enough to be inserted, but remains in place once inserted.

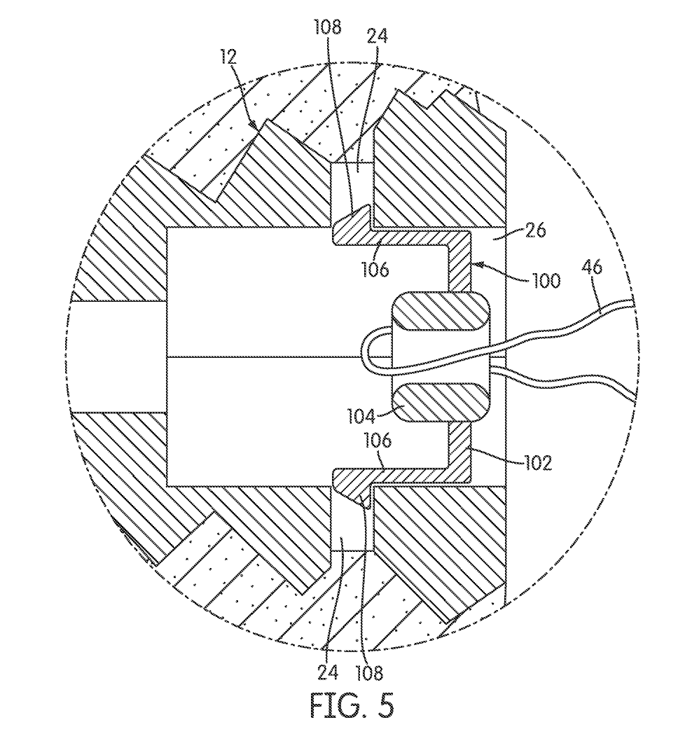

FIG. 5 is a cross-sectional view similar to the view of FIG. 4, but illustrating a different structure, generally indicated at 100, for retaining a suture 46. More specifically, the structure 100 is a clip with a horizontal bar 102 that includes a suture-retaining ring 104 at its center. The bar 102 has a pair of depending prongs 106, each of which terminates in an outwardly-extending barb 108. The clip 100 is approximately the width of the socket 26, but the barbs 108 give it a greater total width. When one slides the clip 100 into the socket, the barbs 108 cause the prongs 106 to deflect slightly inwardly. When the barbs 108 coincide with the holes 24, they snap into place, securing the clip 100.

In the embodiments of FIGS. 4 and 5, if necessary, the bar 28 or clip 100 could be removed from its position by accessing the holes 24 from the exterior and pushing on the pins 30 or barbs 108. This is one advantage of using holes 24 that extend through the full thickness of the head 12 and open on both sides. A tool with inwardly-extending prongs may be used for this purpose.

The anchoring mechanisms of FIGS. 3-5 are useful if the surgeon is using traditional suture material. Embodiments of the invention may also be made for use with other types of sutures and suture anchor technologies. For example, the screw 10 could be used with JUGGERKNOT® bunching-type soft suture anchor technology (Biomet, Inc., Warsaw, Ind., USA). This type of suture includes a polyester sleeve anchoring element that bunches up under tension to retain the suture in place. One would simply place the anchoring end of that type of suture in one of the holes and apply tension, causing the sleeve to bunch such that it cannot pass through the hole 24.

As those of skill will appreciate, while a horizontally-extending hole 24 may be useful and useable to retain a bunching-type suture like the JUGGERKNOT®, if the suture in question is to extend parallel to the long axis of the screw 10, it may be forced to make a 90° turn, which may create undue stress on the suture or bring it into contact with sharp ends of the threads of the head 12, if the head 12 is threaded. Additionally, in other cases, it may be helpful to have a suture extend adjacent to the screw 10, rather than being aligned with its length. For these reasons, in some cases, screws according to embodiments of the invention may include holes that are not perpendicular to the length of the screw. For example, this would allow a suture to hold tissues to bone adjacent to the screw head, which might help the healing of a tendon or ligament to the bone surface.

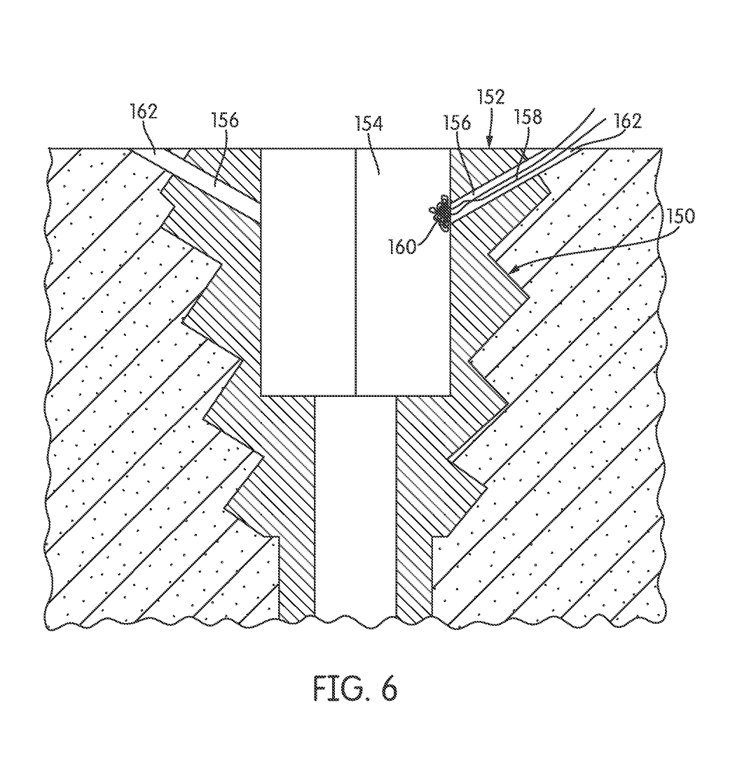

As one example, FIG. 6 is a partial cross-sectional view of the head 150 of a screw 152 according to yet another embodiment of the invention. The head 150 has a hex socket 154, much like other embodiments described above. As shown, a pair of holes or passages 156 extend at an angle to the horizontal through the sidewall of the head 150, opening on both sides. These holes or passages 156 make an acute angle with a long axis of the screw 152 and are mirror images of one another. A bunching suture 158 is shown in one of the holes 156; a sleeve or collection of bunched material 160 created when the suture 158 is tensioned is larger than the hole 156, thus keeping the suture 158 in place.

FIG. 6 also illustrates that in this particular case, the surgeon has created passages 162 in the surrounding bone that are contiguous with and reach the holes 156. This allows the suture 158 to protrude beyond the surface of the bone. These passages may be formed using a drill, a trocar, or another suitable surgical tool.

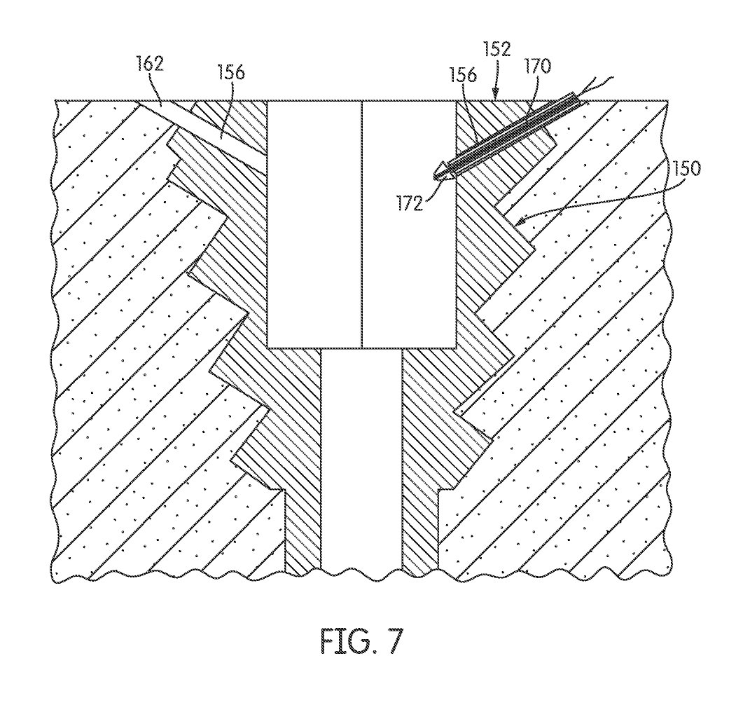

Holes 156 and passages 162 may be used with other types of anchorable sutures. FIG. 7 is another cross-sectional view of the screw 152, illustrating a sleeved anchor suture 170 with spring-loaded arms that have barbs 172 at their ends. The barbs 172 have a greater diameter than the holes 156, and thus keep the suture 170 in the hole 156. Thus, screws according to embodiments of the invention can be made to function with a wide variety of existing suture technologies.

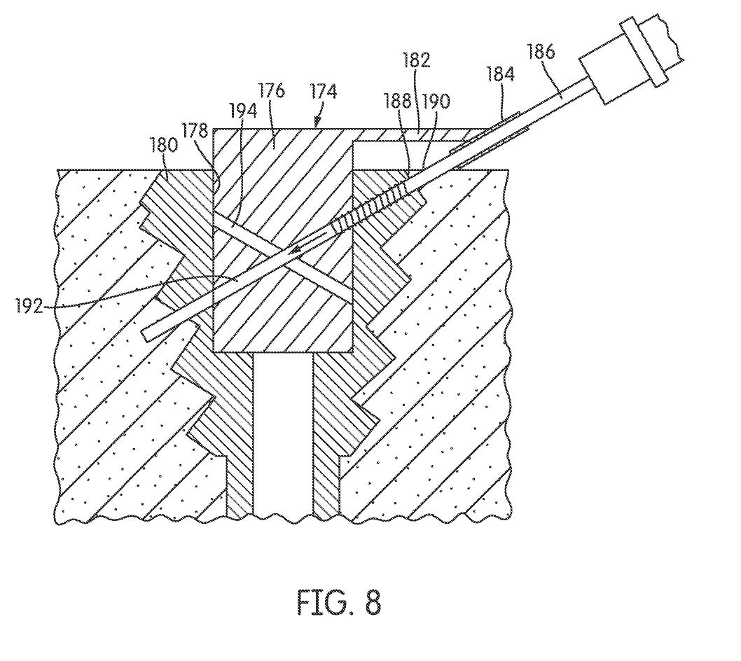

One challenge in using a screw head 150 with angled holes 156 lies in creating the passages 162 in the surrounding bone and tissue that allow the clinician to access and use those holes. FIG. 8 is a cross-sectional view similar to the views of FIGS. 6 and 7, illustrating a method and tooling for creating the passages 162.

More specifically, a tool 174 includes a plug 176 that is sized and adapted to engage the socket 178 of the head 180. The plug 176 would usually be made in the same configuration as a driving tool for the screw head 180. Thus, if the screw head 180 is adapted for a hex driving tool, the plug 176 would be configured as the end of a hex driver would. The plug 176 inserts completely into the socket 178.

The top of the plug 176 is attached to a bracket 182 that extends laterally outward. The bracket 182 supports an angled guide channel 184 that is set at the same angle as one of the angled holes 156. The surgeon would insert a drill bit 186, or another hole-making tool, through the channel 184, thus making a passage 190 through the bone and tissue 188 that is contiguous with the holes 156 and is angled at the same angle as the holes 156. In some embodiments, a small trocar or another type of cutting or punching tool may be used. The plug 176 has passages 192, 194 through its thickness allowing the drill bit 186 or other cutting or punching tool to pass through in order to ensure complete formation of the passages 190. The two passages 192, 194 have opposite slopes from one another, so that they can be used to form one hole on each side of the screw head 180.

Although the tool 174 is primarily useful for creating the passages 162, 190, depending on the material of which the screw head 180 is made, the tool 174 may be used to bore holes through both the bone and the screw head 180 itself. Thus, the tool 174 may be used, in some embodiments, to turn any ordinary surgical screw into a screw according to embodiments of the present invention.

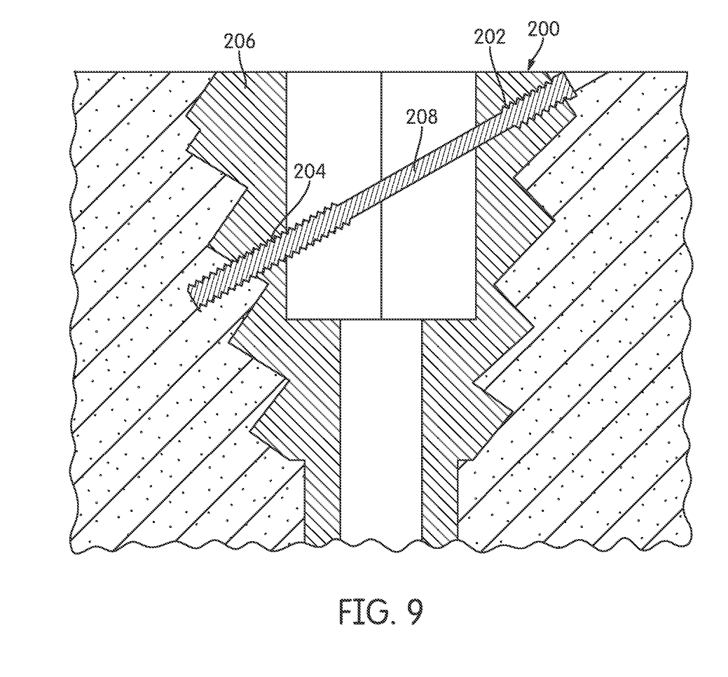

While FIGS. 1-8 illustrate embodiments of the invention in which two holes 24, 156 are positioned opposite one another in a screw head 12, 150, 180. However, that need not be the case. A drill bit 186 can pass through both sides of the screw head 180 in one motion, making two holes that are in line with one another with the same slope. FIG. 9 illustrates this. Specifically, a screw head 200 has two holes 202, 204 in its sidewall opposite and linearly aligned with one another and having the same slope, such that in this case, one hole 202 is above the other hole 204. These two holes 202, 204 may be drilled in the sidewall 206 of the screw head 200 or pre-formed in it. In FIG. 9, these holes 202, 204 are tapped or threaded, and a second screw 208 passes through and is secured in the two threaded holes 202, 204. This kind of configuration may be helpful when fixation of surrounding bone 210 is needed along more than one axis, or when reinforcement in the plane of the holes 202, 204 is desirable.

FIG. 9 also illustrates another aspect of the invention: if the holes 24, 156, 202, 204 of any embodiment are designed or intended to receive a screw 208 or another type of threaded structure, they may be threaded in order to receive and secure that structure. If a structure is threaded, it need only be seated in one of the holes 202, 204 in order to be secured in place; it need not extend through the entire head 200. Moreover, that hole need not be a through-hole; instead, it could be a blind hole. In one embodiment, a suture may be secured to or within a threaded plug, either permanently or by means of structure like the ring 32 of FIGS. 1-4, and a corresponding threaded plug may be inserted into a corresponding threaded hole 202.

The application of the suture anchor aspect of the invention could be widespread in the orthopedic treatment realm. Ligaments hold bone to bone and often need to be secured to bone to allow healing of the ligament in an appropriate position. Some pathological situations require bones to be held in approximation to one another and thus let the ligament heal at appropriate length. A specific application of the invention would be in the ankle, when the anterior talofibular ligament could be reapproximated to the fibula through a suture which would attach to a screw holding a fibular fracture. With medial malleolus fractures of the ankle, a screw head would be an ideal location for further securing of the deltoid ligament. Another application in the ankle would be securing the fibula to the tibia with a bimalleolar ankle fracture with syndesmotic instability, one arm of the anchor would be in the tibia and the other secured to the fibula through fixation screws in the bone or screws in the screwand plate complex often used to hold the fibula in anatomic position. A fracture of the base of the fifth metatarsal could benefit from a suture anchor into the screw head which would help further secure the peroneus brevis attachment often stripped from the bone with the joint capsule in surgical exposure. A small cannulated screw holding a first toe proximal phalanx osteotomy could benefit from a suture anchor which could help reattach the medial joint capsule as well as the abductor hallucis tendon to the bone.

In the upper extremity, elbow fractures might benefit from a suture anchor in a screw holding the olecranon fracture in place; the suture could help secure the triceps back to the olecranon. A number of ligaments can be damaged in complex elbow fracture dislocations and a small cannulated screw with suture anchor attachment could help with reconstruction of the soft tissues as well as the bone repair. In the shoulder, fractures of the proximal humerus might be better fixed with a screw that has an anchor point for reattachment of the rotator cuff to the humeral head. In the wrist, the common scaphoid fracture might benefit from a fixation screw with suture anchors to repair ligaments often concurrently damaged. In the hip, a screw in the greater trochanter can be used as an anchor point to reattach the gluteus medius tendon.

As was described above, screws in general and orthopedic screws in particular are easily stripped, as a result of their size and the force levels involved. Here, the term “stripped” refers to a screw whose socket has been deformed by force or abrasion to the point where it is difficult to turn the screw with a driving tool.

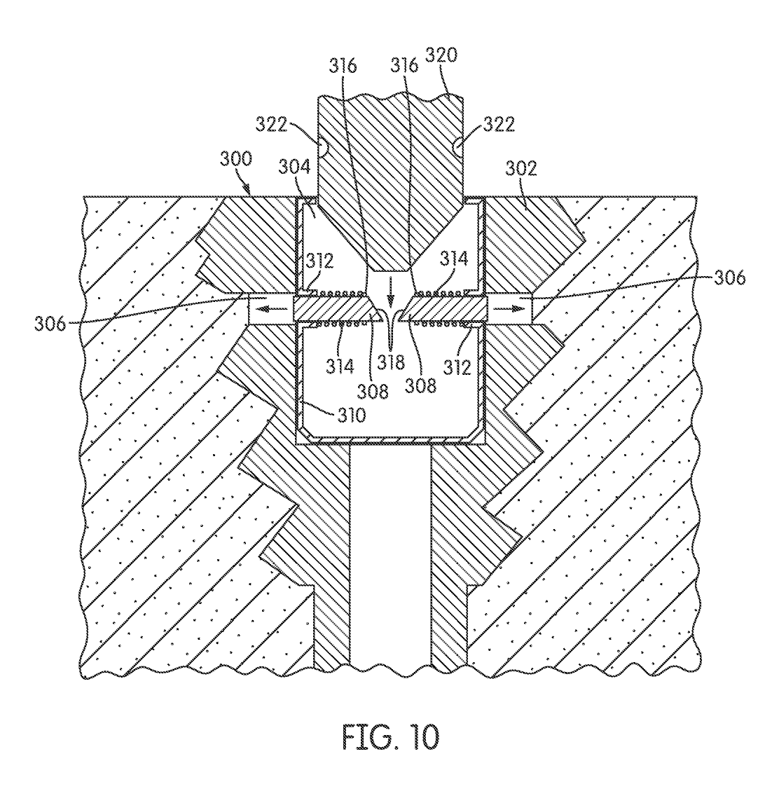

FIG. 10 illustrates a screw, generally indicated at 300, according to yet another embodiment of the invention shown in cross-section in a view similar to the view of FIG. 4. The screw head 302 has a socket 304. The socket 304 has a pair of horizontal holes 306 opposite one another in its sidewall. In this embodiment, respective arms or pins 308 extend from the holes 306 into the center of the socket 304. The socket 304 has a lining 310 that forms guide flanges 312 for the pins 308, constraining the motion of the pins 308 to a horizontal, reciprocating motion in and out of the holes 306. Springs 314 bear between the flanges 312 and a ridge 316 on each of the pins 308. The springs 314 bias the pins 308 out of the holes 306 and toward the extended position shown in FIG. 10.

The pins 308 have angled end faces 318. When a driving tool 320 is inserted into the socket 304, it parts the pins 308 and pushes them aside. The pins then bear against the tool 320 and may insert into pockets 322 in the tool 320. This positive engagement helps to retain the tool 320 in the socket 304 and to prevent the socket 304 from being stripped. Of course, a screw 300 of this type retains compatibility with traditional driving tools, and the extra engagement with the holes 306 may be needed only when the screw head 302 is stripped or there is some other issue.

While the invention has been described with respect to certain embodiments, the description is intended to be exemplary, rather than limiting. Modifications and changes may be made within the scope of the invention, which is set forth in the appended claims.

What is claimed is:

1. A surgical screw, comprising:
a threaded screw head with a socket that defines an interior head sidewall and an exterior head sidewall, the socket being shaped to accept and be driven by a driving tool;
a pair of holes defined opposite one another in the screw head, extending between and opening to the interior and the exterior head sidewalls; and
a shaft connected to the screw head, the extent of the shaft defining a long axis of the screw, at least a portion of the shaft being threaded;
wherein the screw is adapted to be inserted to a point at or below the surface of the bone and to secure two pieces of bone together.

2. The surgical screw of claim 1, further comprising a bar or clip adapted to insert into the pair of holes, the bar or clip carrying suture-attaching structure.

3. The surgical screw of claim 2, wherein the suture-attaching structure comprises a ring or hole.

4. The surgical screw of claim 2, wherein the bar or clip comprises a bar with spring-biased ends that insert into the pair of holes.

5. The surgical screw of claim 2, wherein the bar or clip comprises a clip with depending prong ends.

6. The surgical screw of claim 1, wherein the pair of holes are perpendicular to the long axis of the screw and their openings are aligned with one another.

7. The surgical screw of claim 1, wherein each of the pair of holes makes an acute angle with respect to the long axis of the screw.

8. An orthopedic screw, comprising:
a threaded screw head with a socket that defines an interior head sidewall and an exterior head sidewall, the socket being shaped to accept and be driven by a driving tool;
a shaft connected to the screw head, the extent of the shaft defining a long axis of the screw, at least a portion of the shaft being threaded; and
a pair of holes defined in the screw head, extending between and opening to the interior and the exterior head sidewalls, each of the pair of holes making an acute angle with respect to the long axis of the screw;
wherein the screw is adapted to be inserted to a point at or below the surface of the bone and to secure two pieces of bone together.

9. The orthopedic screw of claim 8, wherein the holes are opposite one another and are mirror images of one another.

10. The orthopedic screw of claim 8, wherein the holes are opposite one another with one hole below the other and interior passageways of both of the holes aligned with one another.

11. The orthopedic screw of claim 10, wherein the pair of holes is threaded.

12. A screw-based anchoring kit, comprising:
a screw, including
a threaded screw head with a socket that defines an interior head sidewall and an exterior head sidewall, the socket being shaped to accept and be driven by a driving tool,
a shaft connected to the screw head, the extent of the shaft defining a long axis of the screw, at least a portion of the shaft being threaded, and
a first passage defined in the screw head, extending between and opening to the interior and the exterior head sidewalls, the first passage making an acute angle with respect to the long axis of the screw,
wherein the screw is adapted to be inserted to a point at or below the surface of the bone and to secure two pieces of bone together; and a guide tool, including
a plug adapted to fit within the socket of the screw,
a support connected to the plug, and
a guide connected to the support, the guide having guide structure positioned to allow creation of a hole or passage adjacent to the screw that is contiguous with the passage in the screw head.

13. The screw-based anchoring kit of claim 12, further comprising a second passage opposite the first passage in the screw head, the second passage being a mirror image of the first passage.

14. The screw-based anchoring kit of claim 12, further comprising a second passage in the screw head, the second passage being located in a position opposite and above or below the first passage, the first and second passages being linearly aligned with one another.

15. The screw-based anchoring kit of claim 14, wherein the first and second passages are threaded.

16. The screw-based anchoring kit of claim 15, further comprising a second screw adapted to be inserted into and secured within the first and second passages.

17. The screw-based anchoring kit of claim 12, further comprising an anchored suture sized and adapted to be secured within the first passageway.

18. The screw-based anchoring kit of claim 17, wherein the anchored suture is a bunching-type anchored suture.

19. The screw-based anchoring kit of claim 17, wherein the anchored suture has spring-biased arms or barbs that pass through the first passageway and engage the screw head along its interior head sidewall.

20. An orthopedic screw, comprising:
a screw head having a socket;
a shaft connected to the screw head and extending therefrom;
a pair of openings provided in the sidewall of the screw head, opening into the socket;
pins inserted into the pair of openings; and
resilient members biasing the pins outwardly, into the socket.
